(19) United States
(12) Plant Patent Application Publication
(10) Pub. No.: US 2010/0043110 P1
(43) Pub. Date: Feb. 18, 2010

(54) Cherry tree named 'Korvik'

(50) ;

(76) Inventor: Vaclav Ludvik, Horice (CZ)

Correspondence Address: STRATTON BALLEW, 213 S 12TH AVE, YAKIMA, WA, 98902, US

(21) Appl. No.: 12/460,904

(22) Filed: Jul. 23, 2009

Related U.S. Application Data
(60) Provisional application No. 61/137,623, filed on Jul. 31, 2008.

(51) Int. Cl. A01H 5/00

(52) U.S. Cl. PLT/181

(57) ABSTRACT

‘Korvik’ is a new and distinct cherry variety (Prunus avium) notable for its round shape and resistance to cracking. ‘Korvik’ is further notable for its productivity on dwarfing rootstocks, and tolerance to Monilia disease (“brown rot”).

### CROSS-REFERENCE TO RELATED APPLICATIONS

This is a Non-Provisional Plant Patent Application based upon Provisional Patent Application No. 61/137,623 and claims priority under 35 U.S.C. 199(e).

### STATEMENT REGARDING FEDERALLY SPONSORED RESEARCH OR DEVELOPMENT

None

### LATIN NAME OF THE GENUS AND SPECIES OF THE PLANT CLAIMED

Prunus avium

### VARIETY DENOMINATION

‘Korvik’

### BACKGROUND AND SUMMARY OF THE VARIETY

‘Korvik’ originated in 1973 as a result of a controlled cross of ‘Kordia’ and ‘Vic’ cherry trees. Asexual propagation by budding was first carried out in 1975 in East Bohemia, Czech Republic. The variety has since been observed over a number of asexually propagated generations, and has been found to remain true to type.

‘Korvik’ is a new and distinct cherry variety notable for its round shape and resistance to cracking. ‘Korvik’ is further notable for its productivity on dwarfing rootstocks, and tolerance to Monilia disease (“brown rot”).

### BRIEF DESCRIPTION OF THE PHOTOGRAPHS

FIG. 1 shows the fruit and leaves of ‘Korvik’; and

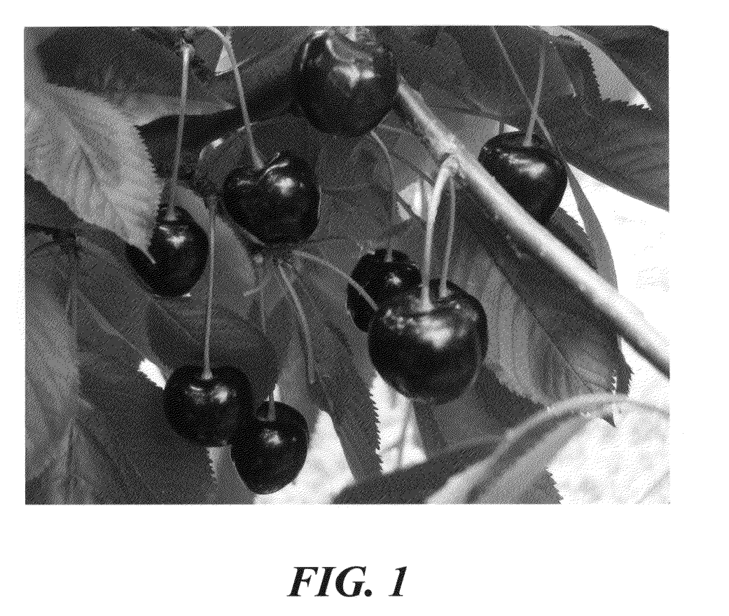

FIG. 2 shows the fruit of ‘Korvik’.

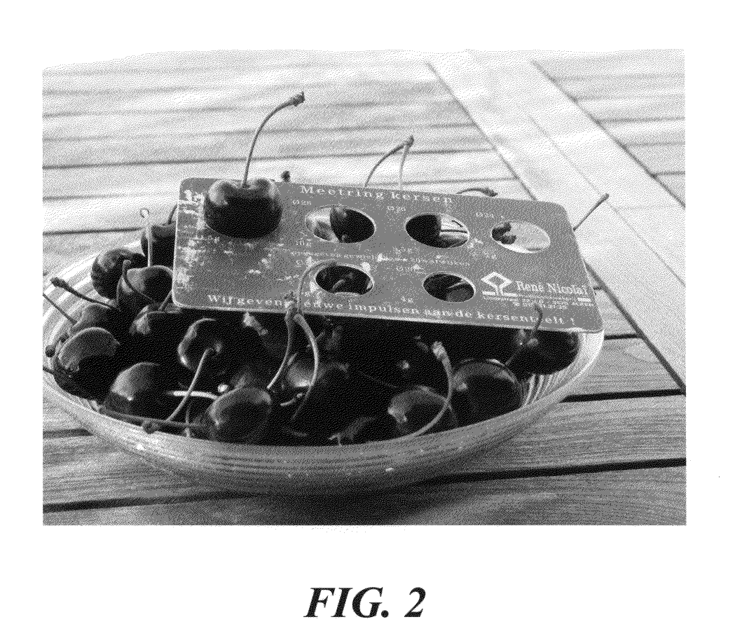

### DETAILED BOTANICAL DESCRIPTION

The following detailed botanical description is based on observations of ‘Korvik’ cherry trees grown at East Bohemia, Czech Republic. Color descriptions refer to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Flowers:
   Time of beginning of flowering.—Late.
Fruit:
   Size.—Large.
   Fruit shape.—Round.
   Color of skin.—Dark Red.
   Color of flesh.—Medium Red.
   Tendency to crack.—Resistant to cracking.
   Firmness.—Medium.
   Time of ripening.—Late.
Orchard performance:
   Disease resistance/tolerance.—Tolerant to Monilia.
   Yield.—Very good yields on dwarf rootstocks.

1. We claim a new and distinct cherry tree substantially as shown and described herein.
